(19) United States
(12) Patent Application Publication
(10) Pub. No.: US 2007/0243239 A1
(43) Pub. Date: Oct. 18, 2007

(54) Patch capable of dispensing a scent

(76) Inventor: Dale E. Lanser, Elkhart Lake, WI (US)

Correspondence Address: WILHELM LAW SERVICE, S.C., 100 W LAWRENCE ST, THIRD FLOOR, APPLETON, WI, 54911, US

(21) Appl. No.: 11/786,102

(22) Filed: Apr. 10, 2007

Related U.S. Application Data
(60) Provisional application No. 60/792,249, filed on Apr. 13, 2006.

(51) Int. Cl. A61L 15/16

(52) U.S. Cl. 424/448

(57) ABSTRACT

A patch is disclosed which is capable of dispensing a scent. The patch includes a pad impregnated with a liquid and the pad is located between a liquid-impermeable barrier layer and a vapor permeable carrier layer. An evaporative interface is situated between the pad and the carrier layer and represents a location where the liquid is transformed into a vapor as it contacts air. A plurality of vaporization ports extend from the evaporative interface to an upper surface of the carrier layer and provide an escape route through which the vapor can escape. The patch is designed to be housed in a package until it is ready to be used. When the package is opened, the patch will emit vapors which are released into the surrounding environment over a desired period of time.

### PRIORITY CLAIM

This application claims priority under 35 U.S.C. 120 from U.S. provisional application Ser. No. 60/792,249, filed Apr. 13, 2006, which is incorporated herein by reference in its entirety.

### BACKGROUND

This invention relates to a patch capable of dispensing a scent. More particularly, this invention relates to a patch that can dispense vapors in the form of an attractant scent, a masking scent and/or a repellant scent. The scent can be an attractant scent, such as a human body fragrance, a perfume, a cologne, a scent used to attract game, such as a fish attractant scent or a wild game attractant scent. Alternatively, the scent can be a masking scent, such as a deodorant. Still another possibility is that the patch can dispense a repellant scent, such as an insect repellant. Another possibility is to design a patch that contains two or more ingredients, each delivering a specific scent or where one ingredient combines with the other ingredient to deliver a single scent.

In the case of insect repellants, both humans and animals can suffer from the attacks of unwanted insects that use the human or animal as a host. The use of the host can be medically neutral such as use as a housing location. In other cases, the insect uses the host as a food supply, such as for sucking blood from the host, commonly known as an “insect bite”.

Many insects carry harmful infective organisms, such as bacteria, which can be transferred from the insect to the host, and which can have a deleterious affect on the host after such transfer. In addition, some insects inject compounds into the host in the process of sucking blood from the host. The result of such injected compounds is commonly realized as a negative skin reaction.

In many cases, the biting/sucking process is painful to the host. In many cases, the skin becomes reddened and raised at the situs of the bite. The host also commonly experiences an itching sensation at the situs of the bite.

Particularly in situations where the host is exposed to multiple such insects, the presence of such insects can be quite distracting, if not painful, and in some cases even emotionally disturbing, to the host.

In the case of livestock such as cattle, the presence and biting of insects on the host animals can measurably reduce the efficiency of growth of the animals, as well as the health of the animals.

Accordingly, substantial efforts have been made to prevent insects from biting both human and animal hosts. Thus, a substantial number of formulas and chemical compositions have been developed which are effective to repel insects from such host bodies, whereby the insects do not land on such host bodies. Since the insects do not land on the host bodies, the insects do not bite the respective host bodies.

There are many known insect repellant formulations that use a variety of insect repellant active materials. See European Patent Applications 97,812 and 97,813, and U.S. Pat. Nos. 4,127,672, 4,756,905, 5,465,685, 5,489,433, 5,565,208, 5,672,337 5,716,602, and 6,646,011.

Depending on the active ingredient selected for the insect repellant, the composition can be useful in repelling such insects as ticks, mites, lice, flies, fleas, mosquitoes, and the like. So long as the insects can be repelled from the host body, the objective of avoiding being bit is accomplished.

Many of the commercially available insect repellant formulations include insect repellant active materials which contain one or more ester, amide or urethane functionalities.

Other commercially available insect repellant formulations contain water insoluble active materials and correspondingly contain solubilizers such as lower monohydric alcohols such as ethanol and isopropanol.

As application methods, it is known to apply the insect repellant to the skin or clothing of a user, from an aerosol spray can. It is also known to carry the insect repellant composition in a cream base, and thus to apply some of the cream to the user's hand and use the hand to spread the cream to other areas of the skin at desired locations. It is further known to pour a liquid insect repellant onto the user's hand and use the hand to spread the liquid to other areas of the skin at desired locations. It is yet further known to impregnate a woven or non-woven cloth or wiper or towelette with an insect repellant composition, and use the cloth or wiper or towelette to apply the insect repellant to the user's skin at desired locations.

For some users, the benefits of the compositions of known insect repellants are at least partially off-set by sensitivity of the skin to the chemical composition of the insect repellant, whereby the skin has an adverse reaction to the composition of the insect repellant. Accordingly, some users are unable to tolerate the chemical composition of known insect repellants, and cannot avail themselves of the benefits of such insect repellants. In other users, the skin reaction is not so severe as to preclude use of the insect repellant on the skin; but the skin reaction is still adverse to the extent it would be desirable that the insect repellant not contact the skin of the user.

Known containers and dispensers are quite bulky in order to contain the insect repellant composition. Typically, the insect repellant is contained in an aerosol can, in a squeeze bottle, or the like. Such containers occupy substantial space to the extent that it is inconvenient to carry such container as a routine component of the user's apparel.

One periodically encounters a situation where insect repellant would be desirable, but wherein a need for insect repellant has not been anticipated, such that no one thought to bring insect repellant with them. For example, an outdoor event unexpectedly lasts into the evening when insects such as mosquitoes are commonly most active. In such situation, it is quite possible that no one has any insect repellant with them, whereby the participants suffer from insect bites.

It would be desirable to provide an insect repellant container/dispenser which is small enough to be readily carried with the user at all times.

It would be further desirable to provide an insect repellant container/dispenser which dispenses the insect repellant in close proximity to the user while not bringing the insect repellant into contact with the user or the user's clothing.

It would also be desirable to provide a patch which would include a scent containing a human body fragrance, such as a perfume or cologne.

It would be desirable to provide a patch which would include a scent for attracting one or more species of fish.

It would be desirable to provide a patch which would include a scent for attracting one or more species of animal.

It would be desirable to provide a package which would contain two or more patches, each individually wrapped and at least two of said patches containing a scent for attracting a certain species of fish or animal.

It would be desirable to provide a package which would contain multiple patches, each containing a unique scent.

### SUMMARY OF THE INVENTION

This invention relates to a patch capable of dispensing a vapor containing a unique scent. The scent can be an attractant scent, a masking scent or a repellant scent. Examples of such scents include but are not limited to: a perfume, a cologne, a deodorant, a fish attracting scent, a wild game attracting scent, etc. The patch includes a pad impregnated with a liquid and the liquid is capable of being vaporized. The pad has a first surface and a second surface. The patch also includes a carrier layer positioned above the first surface of the pad. The carrier layer has an upper surface and a lower surface with the upper surface forming a top outside surface of the patch. The carrier layer also has a periphery. The patch further includes a barrier layer positioned below the second surface of the pad. The barrier layer has a periphery smaller than the periphery of the carrier layer. The patch further includes a fastener secured to at least a portion of the lower surface of the carrier layer. The fastener cooperates with the barrier layer to form a lower surface of the patch. The fastener is capable of releasably attaching the patch to another object. The patch further includes a release layer positioned below the barrier layer and is removably secured to the fastener. The release layer is designed to be removed before the patch is used. The patch also has an evaporative interface situated between the pad and the carrier layer where the liquid is transformed into a vapor upon contact with air. Lastly, the patch includes a plurality of vaporization ports extending from the evaporative interface to the upper surface of the carrier layer through which the vapors can pass.

In another embodiment, this invention relates to a patch capable of dispensing a vapor containing a specific scent. The patch includes a pad impregnated with a liquid and the liquid is capable of being vaporized. The pad has a first surface and a second surface. The patch also includes a carrier layer positioned above the first surface of the pad. The carrier layer has an upper surface and a lower surface with the upper surface forming a top outside surface of the patch. The carrier layer also has a periphery. The patch further includes a barrier layer positioned below the second surface of the pad. The barrier layer has a periphery smaller than the periphery of the carrier layer. The patch further includes an adhesive secured to the lower surface of the carrier layer. The adhesive cooperates with the barrier layer to form a lower surface of the patch. The adhesive is capable of releasably attaching the patch to another object. The patch also includes a repellant layer positioned between the lower surface of the pad and the barrier layer which assist in causing the vapors to exit the patch. The patch further includes a release layer positioned below the barrier layer and is removably secured to at least a portion of the adhesive. The release layer prevents the adhesive from becoming contaminated prior to attachment of the patch to another object. The patch still further includes an evaporative interface situated between the pad and the carrier layer. Lastly, the patch includes a plurality of vaporization ports extending from the evaporative interface to the upper surface of the carrier layer. The plurality of vaporization ports enables the vapors to exit the patch.

In still another embodiment, this invention relates to a patch capable of dispensing a vapor containing a specific scent. The patch includes a pad impregnated with a liquid and the liquid is capable of being vaporized. The pad has a first surface and a second surface. The patch also includes a carrier layer positioned above the first surface of the pad. The carrier layer has an upper surface and a lower surface with the upper surface forming a top outside surface of the patch. The carrier layer also has a periphery. The patch further includes a barrier layer positioned below the second surface of the pad. The barrier layer has a periphery smaller than the periphery of the carrier layer. The patch further includes an adhesive secured to the lower surface of the carrier layer. The adhesive cooperates with the barrier layer to form a lower surface of the patch. The adhesive is capable of releasably attaching the patch to another object. The patch also includes a repellant layer positioned between the lower surface of the pad and the barrier layer. The repellant layer assists in causing the vapors to exit the patch. The patch further includes an attractant layer positioned between the upper surface of the pad and the carrier layer. The attractant layer assists in causing the vapors to exit the patch. The patch further includes a release layer which is positioned below the barrier layer and which is removably secured to at least a portion of the adhesive. The release layer prevents the adhesive from becoming contaminated prior to attachment of the patch to another object. The patch still further includes an evaporative interface situated between the pad and the carrier layer. Lastly, the patch includes a plurality of vaporization ports extending from the evaporative interface to the upper surface of the carrier layer. The plurality of vaporization ports enables the vapors to exit the patch.

In a specific embodiment, the invention relates to a flexible package which defines an insect repellant dispenser in the form of an adhesively mounted patch. The patch includes an adhesively active mounting surface for mounting the patch to a substrate. Vapor ports extend from the opposing surface to a liquid-bearing reservoir in the form of a pad. A barrier layer between the pad and the mount surface protects the surface to which the patch is mounted from coming in to contact with the insect repellant liquid. The patch, with the insect repellant liquid contained therein, is contained in a closed and sealed overwrap packaging structure until used. When the package is opened, vapors are released from the contained liquid insect repellant into the surrounding environment over a desired period of time.

The patch can be attached to any desired substrate such as to a user's skin or clothing, or to a table or other piece of outdoor furniture, or to a surface in a cabin of a boat. In general, the patch can be attached to a substrate surface anywhere insect repellant would otherwise be applied from a different dispensing mechanism, whether from an aerosol can or from e.g. a citronella candle. The patches can operate as generally point sources of insect repellant vapor, much like a citronella candle, but are small enough, and sufficiently convenient, to be mobilized so as to move around with the user, such as on the user's person. Thus, exemplary substrates include: human skin, animal skin, animal hair and animal fur, an article of clothing being worn by a human or by an animal, such as a dog or cat. The substrate can be inanimate, such as a table or other piece of furniture, a patio deck, a boat, or any other structure at or near where insects are expected to become a nuisance to people or animals

An absorbent pad in the patch serves as the reservoir of product to be dispensed. The barrier layer is disposed between the pad and the substrate when the patch is adhesively attached to the substrate. The carrier web is permeable to vapors of the insect repellant liquid or other product to be dispensed. The pad is between the barrier layer and the carrier web, whereby insect repellant vapors are preferentially dispensed outwardly of the patch into the air rather than through the barrier layer toward the mounting surface of the patch. The carrier web, which extends outwardly of the pad and outwardly of the barrier, is coated with a suitable adhesive on the mounting surface side of the web. The adhesive is used to adhesively attach the patch to a substrate to which the patch is mounted. A release sheet is mounted over the substrate side of the patch at the mounting surface of the patch, including over the barrier layer and over the adhesive which is coated onto the carrier layer at locations outwardly of the barrier layer. Additional layers can be used to better retain the insect repellant liquid and/or to assist in controlling the rate of release of insect repellant vapors into the air after the package has been opened and the patch thereby activated.

The packaging, which contains the patch before the patch is activated for use, is effective in preventing pre-mature release of the insect repellant composition from the patch. Thus, the packaging is generally impermeable to the liquid insect repellant, as well as to vapors from the insect repellant liquid. Thus, the packaging can be selected from among single layer structures and multiple layer structures including coated papers, polymeric films, structures which include a metal foil layer or a vapor-deposited metallic coating, and the like.

When it is desired to activate the patch, the package is opened, whereby vapors already present inside the package are largely released to the ambient environment as an initial surge of active material. After the package is opened, the release sheet is pulled off the substrate side of the patch. The patch is then adhesively mounted, if and as desired, to the selected mounting substrate. After the initial opening of the package, and after the initial release of the insect repellant vapor which was contained in the closed and sealed package, vapors are released from the patch through the porous carrier material, by ongoing evaporation of insect repellant or other liquid from the pad, while migration of insect repellant liquid and/or vapor to the substrate is prevented by the barrier layer, in cooperation with the adhesive on the carrier.

### BRIEF DESCRIPTION OF THE DRAWINGS

FIG. 1 shows a cross-section of a patch capable of dispensing a vapor.

FIG. 2 shows a cross-section of a second embodiment of a patch capable of dispensing a vapor.

FIG. 3 shows a cross-section of a third embodiment of a patch capable of dispensing a vapor.

FIG. 4 shows a cross-section of a fourth embodiment of a patch capable of dispensing a vapor.

FIG. 5 shows a top view of a representative patch of the invention.

FIG. 6 shows a pictorial view of person wearing three patches, each containing an insect repellant.

FIG. 7 shows a cross-section of the embodiment shown in FIG. 1 after the enclosing overwrap packaging has been removed, and with release of vapors underway.

The invention is not limited in its application to the details of construction or the arrangement of the components set forth in the following description or illustrated in the drawings. The invention is capable of other embodiments or of being practiced or carried out in other various ways. Also, it is to be understood that the terminology and phraseology employed herein is for purpose of description and illustration and should not be regarded as limiting. Like reference numerals are used to indicate like components.

### DETAILED DESCRIPTION

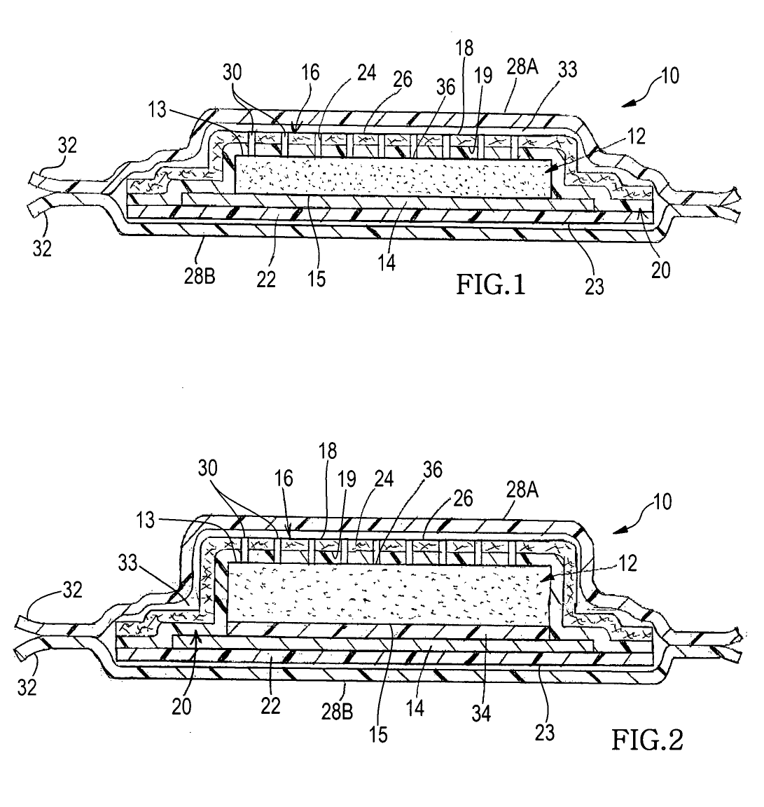

Referring to FIG. 1, a patch 10 is shown which is capable of dispensing a vapor, which has a unique scent, into the atmosphere. The patch 10 includes a pad 12. The pad 12 is enclosed by various layers forming the patch 10 such that the pad 12 is retained within the patch 10. The pad 12 has a first surface 13, a second surface 15 and an outer perimeter. A barrier layer 14 is positioned below the second surface 15 of the pad 12 and extends outwardly beyond the outer perimeter of pad 12. A carrier layer 16 is positioned above the first surface 13 of the pad 12, such that the pad 12 is located between the carrier layer 16 and the barrier layer 14. It should be noted that the first and second surfaces, 13 and 15 respectively, are aligned opposite to one another.

The carrier layer 16 has an upper surface 18, an oppositely aligned lower surface 19, and a periphery. The periphery of the carrier layer 16 extends outwardly beyond the outer perimeter of the pad 12. The barrier layer 14 also has a periphery that is smaller than the periphery of the carrier layer 16. Because of this, the periphery of the carrier layer 16 extends outwardly beyond the outer perimeter of the barrier layer 14. A fastener 20, desirably in the form of an adhesive or an adhesive layer, is present below the carrier layer 16. Desirably, the fastener 20 is an adhesive that is secured to the lower surface 19 of the carrier layer 16 such as by coating, spraying, attaching, painting, slot coating, etc. One skilled in adhesive applications will be aware of these as well as other ways to apply and/or attach an adhesive onto another material or layer.

The fastener 20 can be a glue, an adhesive, an adhesive tape, a hot melt adhesive, a cold melt adhesive, an adhesive having a relatively low tack, an adhesive having a relatively medium tack, an adhesive having a relatively high tack, a co-adhesive, a releasable adhesive, etc. In addition, the fastener 20 can be formed from other materials known to those skilled in the art that may have the properties and/or characteristics of a glue or of an adhesive. As used herein, a “glue” means a strong liquid adhesive or paste obtained by boiling collagenous animal parts such as bones, hides and hooves into hard gelatin and then adding water. By “adhesive” it is meant a substance such as a paste or cement which provides adhesion, a substance that is gummed so as to adhere, or a substance that is sticky. By an “adhesive tape” it is meant a tape lined on one or both sides with an adhesive.

Still referring to FIG. 1, the fastener 20 is depicted as a separate adhesive layer that is secured to the carrier layer 16. Alternatively, the fastener 20 can be applied as a coating, such as by being swirl coated, onto the carrier layer 16. As stated above, the fastener 20 mounts or attaches the carrier layer 16 to the pad 12. In addition, the fastener 20 attaches outwardly of the pad 12 to the barrier layer 14, and outwardly of barrier layer 14 to a release layer 22. The release layer 22 has a periphery essentially coextensive with the periphery of the carrier layer 16. In addition, the release layer 22 is designed to be removed prior to attaching the patch 10 to an object.

Accordingly, the carrier layer 16 is joined by the fastener 20 to the release layer 22. The release layer 22 is positioned below the barrier layer 14. The release layer 22 overlies the barrier layer 14 and forms a bottom outside surface 23 of the patch 10 before the patch 10 is placed into use. The barrier layer 14 is thus located between the release layer 22 and the pad 12. The release layer 22 is removably secured to the fastener 20 and is designed to be removed before the patch 10 is used. The outer perimeters of the release layer 22, the fastener or adhesive layer 20, and the carrier layer 16 are generally coextensive, and generally define the outer perimeter of patch 10. A plurality of vaporization ports 24 extend through the carrier layer 16 and the fastener or adhesive layer 20 at locations on the upper side of the pad 12 and between the pad 12 and the top outside surface 26 of the patch 10. Each of the plurality of vaporization ports 24 forms a continuous passageway through both the fastener or adhesive layer 20 and the carrier layer 16. Each of the plurality of vaporization ports 24 has a restrictive or small opening through which only vapors can pass. In other words, the vaporization ports 24 are sized such that the contained liquid to be dispensed is unable to pass through the plurality of vaporization ports 24.

The vaporization ports 24 thus provide transport paths for egress of vapors, i.e. perfume, cologne, insect repellant, etc. from the pad 12 toward the outside surface of the patch 10, and ultimately into the ambient air in the vicinity of the patch 10.

Still referring to FIG. 1, the patch 10 will be explained as an insect repellant patch solely for the purpose of describing one embodiment of this invention. In the patch 10, the pad 12 is impregnated with a desired quantity of a liquid, for example an insect repellant composition, which can vaporize at ambient conditions. Ambient conditions are commonly present when insects, which are to be repelled, are present. Thus, the insect repellant composition should produce a desired quantity of insect repelling vapors, at a desired rate, for a desired period of time, at ambient temperatures of, for example, about 60 degrees Fahrenheit (F) to about 120 degrees F.

The vaporization rate is in general affected by certain ambient conditions, such as temperature of the insect repellant liquid, temperature of the ambient air, humidity of the ambient air, and the like. The vaporization rate is also affected by the composition of the insect repellant liquid, as well as by the structures, compositions, and locations of the respective layers in the patch 10. Thus, while ambient conditions are beyond the control of the manufacturer or user of the patches 10, the vaporization rate can be controlled to a limited extent by the design and structure of the patch 10 as well as by the composition of the liquid which is impregnated into the pad 12.

Packaging over wrap layers 28A, 28B overlie the top and bottom surfaces of the patch 10, and hold the patch 10 in a closed and sealed environment until such time as the patch 10 is to be used. It should be noted that various types of packaging and packaging material that is known to those skilled in the art, can be used to form the outside wrapper, over wrap layers 28A and 28B or the package.

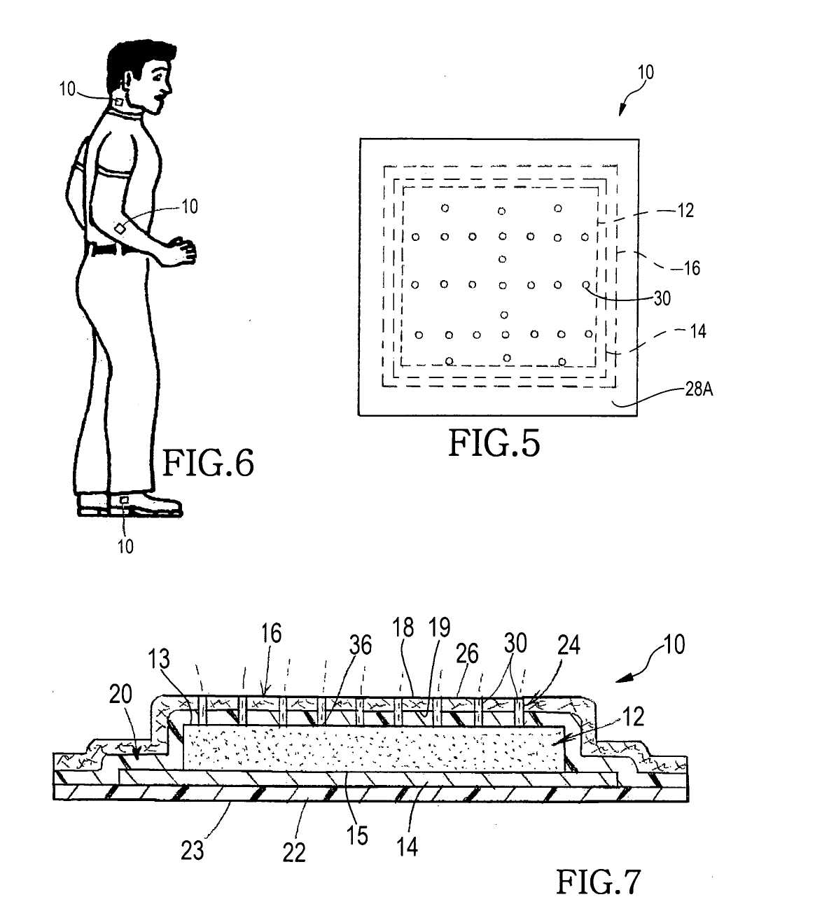

The pad 12 can be manufactured from a wide variety of materials in a wide variety of sizes, and in a wide variety of shapes such as rectangular, circular, oval, trapezoidal, T-shape, I-shape, hourglass shape, etc. A generally square-shaped pad 12 is illustrated in FIG. 5. The size and the liquid-holding capacity of the pad 12 should be compatible with the size of the intended patch 10. The anticipated liquid loading of the pad 12 can be varied in order to provide insect repellant vapors of a desired effective intensity for an acceptable period of time. Thus, to at least some degree, the selection of the pad 12 properties depends on the performance parameters, such as vaporization rate and rate/time relationship, as well as the absolute quantity of insect repellant vapors which are to be released in a given period of time. In addition, the area/space which is to be protected by a given patch 10 further dictates liquid-holding capacity, release rate, rate deterioration with time, and the like which must be designed into a given patch 10.

The pad 12 suitably comprises a matrix of hydrophilic fibers, such as a web, or webs, of cellulosic fibers, open cell foam, closed cell foam, gauze padding, paper/tissue layers, nonwoven layers, and the like, as well as blends and mixtures of such and other absorbent compositions. Whatever the composition of pad 12, the material of the pad 12 should be such that the pad 12 readily absorbs the insect repellant liquid when the liquid is injected or otherwise applied to the pad 12. Yet the pad 12 must not hold the liquid so tightly as to preclude evaporation of a substantial amount of the liquid from the pad 12 in the process of releasing the insect repellant into the ambient environment in its desired use. Thus, the pad 12 should have a relatively high liquid holding capacity, expressed in terms of milliliters of liquid per cubic centimeter of the pad material. But it should also readily release the insect repellant liquid when exposed to ambient air conditions. So the pad 12 liquid holding parameters are selected as a balance of overall liquid-holding capacity and ability of the pad 12 to release the insect repellant into the ambient air.

Regarding foam materials which can be used to form the pad 12, there can be mentioned open-cell polyurethane foams, closed-cell polyurethane foams, open cell polyethylene foams, closed cell polyethylene foams, and polypropylene foams.

If and where cotton gauze is used in constructing the pad 12, a suitable number of layers are used to develop the desired liquid holding capacity.

If and where cellulose fibers are used, the quantity of fibers, the fiber structure, and the inter-relationship of the fibers to each other, are all taken into consideration in designing the pad 12. Similarly, where non-woven fiber materials are used in fabricating the pad 12, the overall liquid holding capacity, the release rate, and release mechanism are all taken into consideration in designing the pad 12. Those skilled in the arts of personal care absorbent technology are well aware of various structures which can be employed in absorbing and releasing various liquids, and so can design suitable pads 12, without further ado, for the pad 12 to be used in the patch 10.

For example, one can use synthetic, polymeric, or meltblown or natural fibers or a combination of wood pulp fluff, synthetic fibers, polymeric fibers, meltblown fibers, and/or natural fibers. A given fiber type can be selected for structural reasons as well as for reasons of absorbency. Where a fiber is desirably selected for structural reasons but has less than the needed absorbency, such fibers can be treated with various e.g. surfactant treating materials in order to enhance absorbency of the fiber or a corresponding fiber matrix.

The pad 12 can comprise a laminate of various ones of the recited materials, or can comprise other suitable structure operative to maintain the integrity of the pad 12, to effect the desired absorption and release properties at desirable locations in the.

Further, the pad 12 can be configured with areas of relatively higher liquid holding capacity and areas of relatively lower liquid holding capacity, areas of relatively higher vapor release rates and relatively lower vapor release rates. Such diversity in the pad 12 can be affected by fabricating the pad 12 from a multiplicity of layers, each having a salient liquid handling property and a desirable location for performance of such salient property.

The barrier layer 14 can be manufactured from a wide variety of materials in a wide variety of sizes, and in a wide variety of shapes such as rectangular, circular, oval, trapezoidal, T-shape, I-shape, hourglass shape, etc. In general, the material, and layer thickness, for the barrier layer 14 are selected such that the barrier layer 14 can serve as an effective barrier to transmission of the insect repellant material, carried in pad 12, from the pad 12 to the user's skin. Thus, it is critical that the barrier layer 14 be imposed between the pad 12 and the user's skin at all locations represented by an image of the pad 12 projected onto the wearer's skin. In addition, the insect repellant liquid should not be able to readily migrate around an outer edge of the barrier layer 14, such as at the outer perimeter of the barrier layer 14.

A generally square-shaped barrier layer 14 is illustrated in dashed outline in FIG. 5. The size and shape of the barrier layer 14 should generally compliment the size and shape of the pad 12. Thus, the barrier layer 14 should extend outwardly, at all locations along its perimeter, beyond the outer perimeter of the pad 12 at the respective locations. Since the barrier layer 14 extends outwardly beyond the outer perimeter of the pad 12, the only way for the liquid in the pad 12 to reach the user's skin is for the liquid to migrate outwardly of the pad 12 to the outer edge of the barrier layer 12, and thence toward the skin.

Referring again to FIG. 1, the fastener or adhesive layer 20 is in direct surface-to-surface contact with the barrier layer 14 about the outer perimeter of the pad 12.

Accordingly, the fastener or adhesive layer 20 serves as a barrier to migration of the insect repellant outwardly to the outer edge of the barrier layer 14. With liquid migration, outwardly to the outer edge of the barrier layer 14, effectively blocked by the fastener or adhesive layer 20, with liquid migration directly from the pad 12 toward the skin effectively blocked by the barrier layer 14, the insect repellant liquid is effectively blocked from reaching the user's skin, whereby the patch 10 can be employed on the user's skin, as desired, without the insect repellant liquid coming into contact with the skin. Thus, since the insect repellant liquid never comes into contact with the user's skin, even those who have skin which is sensitive to insect repellant liquid, when applied by conventional methods, can use conventional-type insect repellants liquids when such insect repellants are loaded into a patch 10 of the invention.

The composition of the barrier layer 14 can be any material which can serve as an effective barrier to movement of the insect repellant liquid, which is to be used in pad 12, to the surface of the user's skin. Thus, selection of the composition of the barrier layer 14 is desirably delayed until the composition of the insect repellant liquid is determined. And while small amounts of migration of the insect repellant liquid is commonly tolerated, no more than a minimal amount is acceptable to the concept of the invention. Thus, the barrier layer 14 desirably stops substantially all of the insect repellant liquid from migrating from the pad 12 to the bottom outside surface 23 of the patch 10. Similarly, any portion of the barrier layer 14 which is expected to come into contact with the skin of the user must be skin-friendly and not irritating to the skin.

In selecting materials which can be used in fabricating the barrier layer 14, there can be mentioned, for example and without limitation, metal foil such as aluminum foil, tin foil, carbon film, silver/carbon film, coated papers, plastic films, and the like. Aluminum foil or tin foil can be, for example and without limitation, about 0.5 mils thick to about 2 mils thick. Carbon film is actually a polymeric vinyl film coated with a layer of carbon. Silver/carbon film has a vinyl substrate which is coated with a layer of carbon and a layer of silver. Such films are commercially available in thicknesses of 2 mils, 4 mils, and 6 mils, any of which can be used so long as they do not detract from the desired flexural properties of the resultant patch 10. Other polymeric films can be used such as, for example and without limitation, polyethylenes, polypropylenes, vinyls, polyesters, nylons, and the like.

The barrier layer 14 can be comprised of multiple layers such as, for example, a polymeric substrate layer having a vapor-deposited metallic layer, such as for example aluminum-coated polyethylene or polyester or nylon. Optionally, the barrier layer 14 comprises a polymeric substrate coated with a metal foil or vapor deposited metallization layer, wherein the polymeric substrate forms the bottom outside surface 23 of the patch 10. Thus, the metal portion of such structure is between the polymeric layer and the pad 12. Correspondingly, the polymeric layer provides that portion of the bottom outside surface 23, defined by the barrier layer 14, which is in contact with the user's skin. Such polymeric material is desirably compatible with extended skin contact. As exemplary of such polymeric materials, there can be mentioned various ones of the polyolefins such as polyethylene, polypropylene, ethylene propylene copolymers, vinyls, polyesters, nylons, and the like.

It is required that the respective barrier layer material effectively prevent the insect repellant from migrating through the barrier layer 14 and reaching the wearer's skin.

The barrier layer 14 is desirably thin, such as about 0.5 mils thick to about 2 mils thick. In any event, the barrier layer 14 must be sufficiently flexible as to enable ordinary flexing of the patch 10 in cooperation with normal movement and flexing of the skin where the patch 10 is adhesively attached to the user's body.

The carrier layer 16 can be manufactured from a wide variety of materials in a wide variety of sizes, in a substantial variety of thicknesses, and in a wide variety of shapes such as rectangular, circular, oval, trapezoidal, T-shape, I-shape, hourglass shape, etc. A generally square-shaped carrier layer 16 is illustrated in FIG. 5. The size, shape, and thickness of the carrier layer 16 should generally complement the size and shape of the pad 12. Thus, the carrier layer 16 should extend outwardly, at all locations along its perimeter, beyond the outer perimeter of the pad 12 at the respective locations, and generally beyond the barrier layer 14. The carrier layer 16 extends horizontally outwardly beyond the outer perimeter of the pad 12 and the outer perimeter of the barrier layer 14. Since the carrier layer 16 is coated over substantially its entire surface with the fastener or adhesive layer 20, once the release layer 22 is stripped away from the patch 10, the fastener or adhesive layer 20, to the extent it extends beyond the barrier layer 14 and the pad 12, is exposed. The exposed adhesive layer 20 can be used to mount or attach the patch 10 to any desired substrate. The substrate can be a user's skin or clothing, or any other desired inanimate object. Examples of inanimate objects include but are not limited to: shoes, sneakers, hats, baseball caps, coats and jackets, rain gear, clothing, especially outer clothing, such as a shirt, a pair of pants, socks, gloves, etc.

The carrier layer 16 and the barrier layer 14 can both be formed from a liquid and vapor impermeable material. When the carrier layer 16 is so formed, it will become vapor permeable when the plurality of vaporization ports 24 is formed therein.

Referring now to FIGS. 5 and 7, a plurality, e.g. a matrix, of spaced vaporization ports 24 extend through the carrier layer 16 and in registration through the fastener or adhesive layer 20. A given representative vaporization port extends along a constant path from the surface of the pad 12 to the top outside surface 26 of the patch 10. In order to effect the apertures in the fastener or adhesive layer 20, and registration of such apertures in the fastener or adhesive layer 20 with respective apertures in the carrier layer 16, thus to make vaporization ports 24, the adhesive material which defines the fastener or adhesive layer 20 is applied to the carrier layer 16. After the fastener or adhesive layer 20 has been applied to the carrier layer 16, the combination of the carrier layer 16 and the fastener or adhesive layer 20 is then applied to pad 12.

The composition of the carrier layer 16 can be any material which can serve as an effective barrier to wholesale loss of the insect repellant liquid in liquid form during use of the patch 10, while having sufficient permeability to vapors of the insect repellant to not obstruct movement of insect repellant vapors out of the patch 10 after the patch 10 is activated by removal of the enclosing packaging overwrap materials 28A and 28B.

In selecting materials which can be used to fabricate the carrier layer 16, a wide variety of sheet materials and/or films can be used. Such sheet materials are commonly considered to provide barriers to migration of vaporizing liquid material. Such sheet materials can include metal foils such as aluminum foil, tin foil, carbon film, silver/carbon film, or the like. Aluminum foil or tin foil can be, for example and without limitation, about 0.5 mils thick to about 2 mils thick. Carbon film is actually a polymeric vinyl film coated with a layer of carbon. Silver/carbon film has a vinyl substrate which is coated with a layer of carbon and a layer of silver. Such films are commercially available in thicknesses of 2 mils, 4 mils, and 6 mils, any of which can be used so long as they do not detract from the desired flexural properties of the resultant patch 10. Other polymeric films can be used such as, for example and without limitation, polyethylenes, polypropylenes, vinyls, polyesters, nylons, and the like. The only requirement is that the material selected for the carrier layer 16 should effectively prevent the liquid insect repellant from passing through the carrier 16 and reaching the wearer's skin.

Such materials can be made compatible with passage of such vapors by fabricating such materials by forming holes, slits, cuts, and the like, in thin-sections such films or foils, or sheets, in order to facilitate passage of the corresponding insect repellant vapors, but not the liquid. In such case, the carrier layer 16 functions as a relatively highly-effective barrier at all locations where the carrier layer material is not penetrated for purpose of enabling passage of vapors. FIG. 5 illustrates such a structure where apertures 30 extend through the carrier layer 16 and through the fastener or adhesive layer 20 and over a substantial portion of the area of the top outside surface 26 of the pad 12. However, no apertures 30 are present where the carrier layer material is otherwise substantially less permeable to the insect repellant vapors at the unperforated locations, such as outwardly of the perimeter of the pad 12. Alternatively, the apertures 30 can be formed through out the entire surface area of the carrier layer 16, if desired, such as to reduce manufacturing cost.

In the alternative, the carrier layer 16 can be made of a relatively permeable sheet material/web such as a natural-fiber non-woven, or a synthetic-fiber non-woven, or a natural or synthetic fiber woven web material. Such carrier layer 16 sheet/web material is inherently reticulated such as by means of the fibrous construction of the web material whereby randomly-spaced or evenly-spaced apertures 30 extend through the sheet/web material and are located about the entirety of the surface area of the carrier layer material. The apertures 30 formed in the carrier layer 16 thus define the locations of respective vapor ports to the extent corresponding apertures are defined in the underlying fastener or adhesive layer 20. In some instances, the sizes of the apertures 30, and the viscosity of the adhesive when applied to the carrier layer web, are such as to inherently provide corresponding apertures in the fastener or adhesive layer 20. In other instances, the apertures 30 can be defined mechanically or otherwise after the adhesive has been applied to the carrier layer web material.

The composition of fastener or adhesive layer 20 can be any material which can adhesively bond the carrier layer 16 to the pad 12 and can also adhesively bond to the barrier layer 14, and an underlying substrate such as the skin of a user. In addition, the adhesive must bond releasably to the material of the release layer 22. The material of the fastener or adhesive layer 20 must be skin friendly, such that the e.g. chemical composition of the adhesive does not negatively affect the health of the skin of the user. Thus, the adhesive must be a “skin-friendly” adhesive, and must satisfy all toxicity requirements related to application of a chemical composition to human skin. Typically, a medical grade adhesive is used. In addition to the above, the adhesive must provide a level of tack which will enable the patch to be mounted to a wide variety of substrates as a contact adhesive. Suitable and exemplary such adhesives are available from 3M Company, St Paul, Minn., under the designations “3M 9472 Adhesive” and “3M 9477 Adhesive”.

The composition of the release layer 22 can be any material which can releasably bond to the fastener or adhesive layer 20, and which is neutral and non-reactive with respect to the barrier layer 14, and which does not transport therethrough any material from either the barrier layer 14 or the fastener or adhesive layer 20. Thus, there can be mentioned a wide range of papers and plastic films which are known for their properties as releasing sheet materials, which either inherently have a low tack with respect to the fastener or adhesive layer 20, and do not adhere permanently to the barrier layer 14, or are coated to provide a release characteristic to such sheet materials. The release layer 22 commonly comprises a base substrate material of either paper or plastic, which is coated with a release coating such as a silicone coating, so as to impart to the resultant structure a release characteristic by which the release layer 22 is readily released from both the barrier layer 14 and the fastener or adhesive layer 20.

In FIGS. 1-4, the top outside surface 26 of the patch 10 is defined by the carrier layer 16. By contrast, the bottom outside surface 23 is defined collectively by the barrier layer 14 and an outer perimeter portion of the fastener or adhesive layer 20 as supported by the carrier layer 16. The entirety of the bottom outside surface 23 is typically overlain by, and covered by, the release layer 22 until such time as the patch 10 is to be placed into use.

FIG. 5 shows a typical top view of a patch 10 of this invention, illustrating the relative placement and registration of the pad 12 inside the outer perimeter of the barrier layer 14, and the barrier layer 14 inside the outer perimeter of the carrier layer 16. FIG. 5 also shows registration of the vaporization ports 24 over the pad 12, and not outwardly of the projected image of the pad 12. In other embodiments, vaporization ports 24 can be located outwardly of the projected image of the pad 12.

Referring now to FIG. 6, three patches 10 are depicted attached to various parts of an adult human and his clothing. FIG. 6 illustrates the patches 10 in locations most commonly exposed to biting insects during the season when insects are most commonly active. Thus, the patches 10 are shown applied to the neck, an arm, and an ankle. At the neck and arm, the patches 10 are shown applied directly to the user's skin. At the ankle, the patch 10 is shown applied to the user's shoe. Any of the patches 10 could as well have been shown applied to the user's cloth apparel. The number of patches 10 needed for a given application depends on the size of the patch 10, the quantity of liquid insect repellant in the pads 12, and the vaporization rate in terms of cubic centimeters of liquid evaporated per hour or other unit of time. A relatively higher vaporization rate suggests a relatively smaller number of patches 10, such as one patch 10 for a limited area to be protected. A relatively lower vaporization rate suggests a relatively greater number of patches 10, optionally targeted to specific smaller and spaced targets of vulnerability. A relatively greater number of patches 10 can be placed close enough together to provide overlapping coverage of an area so as to collectively protect the overall area of interest. In the implementation suggested in FIG. 6, three patches 10 are placed strategically at the areas of the body which most commonly suffer from mosquito bites but with sufficient vapor quantity to provide overall protection to all areas of the body of that user.

In selecting materials which can be used in fabricating the barrier layer 14, there can be mentioned, for example and without limitation, a wide variety of sheet materials and/or films which are commonly considered to provide barriers to migration of vaporizing liquidous material. As such sheet materials there can be mentioned for example and without limitation metal foils such as aluminum foil, tin foil, carbon film, silver/carbon film, or the like. Aluminum foil or tin foil can be, for example and without limitation, about 0.5 mils thick to about 2 mils thick. Carbon film is actually a polymeric vinyl film coated with a layer of carbon. Silver/carbon film has a vinyl substrate which is coated with a layer of carbon and a layer of silver. Such films are commercially available in thicknesses of 2 mils, 4 mils, and 6 mils, any of which can be used so long as they do not detract from the desired flexural properties of the resultant patch. Other polymeric films can be used such as, for example and without limitation, polyethylenes, polypropylenes, vinyls, polyesters, nylons, and the like. The only requirement is that the respective layer material should effectively prevent the insect repellant from migrating through the barrier layer 14 and reaching the wearers skin.

The collective thicknesses of the respective layers provides an overall thickness and stiffness to the patch 10 which leaves the patch 10 sufficiently flexible to readily conform to changing contours of a wearer's skin as the wearer goes about his usual activities. Thus, the patch 10 must be quite flexible considering the wide variety of activities in which people engage. In addition, the adhesive tack must be sufficiently aggressive to not be unintentionally dislodged from the wearer's skin during the contemplated normal use life of the patch 10. However, the adhesive tack must not be so aggressive as to interfere with complete removal of the patch 10 from the skin when and as desired, including removal of substantially all of the adhesive. Thus, the adhesive must have a substantial adhesion to the user's skin, but must at the same time have a greater affinity for the carrier layer and/or the barrier layer 14 so as to peel from the skin rather than from the carrier layer 16 and barrier layer 14 when the patch 10 is removed from the skin.

The liquid-holding capacity of the pad 12 should be compatible with the projected area of the intended patch 10 and the anticipated liquid loading of the pad 12 which is required in order to provide insect repellant vapors of an effective intensity for an acceptable period of time over a desired area, at specified ambient conditions. Thus, to at least some degree, the selection of pad properties depends on the performance parameters, such as vaporization rate and rate/time relationship, as well as the absolute quantity of insect repellant vapors which are to be released from the patch in a given period of time. In addition, the area/space which is to be protected by a given patch further dictates liquid-holding capacity, release rate, rate deterioration with time, and the like, all of which must be designed into a patch 10 for a given implementation.

The patches 10 can be made on a wide variety of conventional web converting machines. For example, the fastener or adhesive layer 20 can be applied to an endless web of material representing the carrier layer 16. The pads 12 are placed on the adhesive at spaced and registered locations on the adhesive-coated carrier web. Pieces of the barrier layer 14 can be cut from a continuous in-feed web of the respective material, and are placed in registration on the pads 12. A release layer web is fed over the entirety of the carrier web 16, and pressed against the contact adhesive which extends, as part of the carrier layer 16 coated composite, outwardly beyond the outer perimeter of the barrier layer pieces, thus bonding the composite structure, and its respective elements, to each other. Individual patches 10 are then cut from the thus-fabricated composite web structure. The individual patches 10 are spaced from each other and are passed between a pair of in-feed packaging overwrap webs. The overwrap webs are brought into enclosing relationship with respective ones of the patches 10 and are sealed closed.

In the manufacturing process, insect repellant liquid is applied to the pad 12 prior to enclosing the patches 10 in the enclosing packaging 28A and 28B. For example, in the illustrated process, the insect repellant liquid can be applied to the pad 12 before the pad 12 is applied to the carrier layer 16, or after the pad 12 is applied to the carrier layer 16 and before the pad 12 is covered by the barrier layer pieces; or the insect repellant liquid can be injected into the pad 12 through the carrier layer 16 after the patch 10 is fully assembled but before the patch 10 is enclosed in the packaging materials 28A and 28B.

In another exemplary process, the patch 10 can be built by using an endless web of the release layer 22 as a substrate on which to build patches 10. Spaced barrier layer pieces are placed on the release layer 22. The patches 10 are placed on the barrier pieces. Liquid insect repellant is applied to the pad 12. A web of the carrier layer material, coated with material of the fastener or adhesive layer 20 is overlaid over the web of release layer material, the barrier pieces and the pads 12, and is pressed against the composite thus to adhesively mount the carrier web to the remaining elements of the patch 10. Any desired fabricating of the apertures 30 in the carrier web 16 and/or in the fastener or adhesive layer 20 is typically done before the coated carrier web is brought into assembly with the remaining elements of the patch 10. The thus-assembled web structure is then cut into individual patches 10, is over wrapped with packaging material 28A and 28B, and is closed and sealed inside the packaging material 28A and 28B. Each patch 10 is thus individually packaged in its own packaging material so as to be separated from all other patches 10. The packages can be formed to be both liquid and vapor impermeable. While two or more such patches 10 can be packaged in a given package, a typical package contains a single patch 10. Multiple such individually-wrapped patches 10 are combined for shipping purposes into conventional shipping containers.

The patch 10 is received by a user in a closed and sealed packaging structure, defined by e.g. packaging overwrap layers 28A, 28B. Overwrap layers 28A, 28B can be e.g. polymer coated paper, plastic film, polymer/foil/paper composite, or the like, or other packaging structure, so long as the resultant packaging structure effectively prevents escape of vapors of the contained insect repellant from the patch, during its expected shelf life of the product, until the package is opened.

The packaging represented by FIGS. 1-4 can be fabricated so that overwrap layers 28A, 28B loosely contain the patch 10. In this packaging environment, the package generally embodies a confined space 33 located between the packaging material layers 28A, 28B and the patch 10. In the packaged embodiments illustrated, the confined space 33 carries an initial amount of vapor of the insect repellant liquid which is contained in the pad 12. The vapor pressure in the confined space 33 is in general equilibrium with the vaporization capability of the liquid insect repellant material in the pad 12.

When the user desires to place the patch 10 into use, he opens the package such as by grasping pull tabs 32 located or formed on the over wrap layers 28A, 28B and peels layers 28A, 28B apart from each other, thus opening the package. At that point, the vapors which are extant in the confined space 33 are immediately released, as an initial surge of protective insect repelling vapors, into the air around the patch 10.

This reduces the vapor concentration at the vaporization ports 24, and at evaporative interface 36 whereby the liquid concentration of the insect repellant in the pad 12 drives further expression of insect repellant vapors from the pad 12, through vapor ports 24, and into the environment surrounding the pad 12, thus providing an ongoing expression of insect repellant vapors from the patch 10 through the period of its useful life. The evaporative interface 36 is a demarcation below which liquid exists in the pad 12 and above which vapor exists in the carrier layer 16.

If desired, the patch 10 can be placed on a surface of an inanimate object. This will allow the vapors from the patch 10 to effectively provide insect repellant properties to the immediate area. The size of the effectively protected immediate area depends on a number of factors. Some of those factors relate to the vaporization rate of the patch 10. Other factors depend on the air environment such as the amount of air movement such as wind.

In another set of embodiments, not shown, a second release layer is mounted or attached over the carrier layer 16, and particularly over the vaporization ports 24, thus to close off, or reduce, the release of insect repellant vapors from the patch 10 and into the confined space 33, until the package is opened by removing the overwrap packaging materials 28A and 28B. The composition of the second release layer, if and as used, can be any sheet material which can be mounted on the carrier layer 16. The second release layer functions to effectively limit, prevent or retard the release of vapors into the confined space 33. By so acting, the quantity of insect repellant vapors present in the confined space 33 is substantially reduced when the packaging, containing the patch 10, is opened. The second release layer is also compatible with being removed at least from over the vapor ports 24 when the patch 10 is to be placed into use. Thus, there can be mentioned many of the same materials which are compatible with being used as the release layer 22, namely a wide range of papers and plastic films, which are known for their properties as releasing sheet materials and which do not adhere permanently to the barrier layer 14, or are coated to provide a release characteristic to such sheet materials. The second release layer can comprise a base substrate material of either paper or plastic, which is coated with a release coating such as a silicone coating, so as to impart to the resultant structure a release characteristic by which the second release layer is readily released from carrier layer 16.

In such a structure, since the quantity of insect repellant vapors in the confined space 33 is limited by the second release layer, the quantity of vapor available for initial release when the packaging material is removed is substantially less. As a result, the quantity of insect repellant vapor which is released when the packaging material is removed is reduced accordingly. However, the additional barrier to escape of insect repellant vapors can be useful in extending shelf life of the packaged patch.

In yet another embodiment, not shown in the drawings, the packaging over wrap layers 28A, 28B are closed tightly about the patch 10 such as by a vacuum packaging process, thereby eliminating the confined space 33 located between the patch 10 and the packaging materials. With the confined space 33 thus eliminated, there is no reservoir of initial vapors to be released and the cost of the second release layer is avoided.

Full benefit of the efficacy of the patch 10 in protecting a person or animal is achieved when the patch 10 is mounted to the host, so as to move about as the host moves about. To that end, the user grasps the release layer 22 and pulls the release layer 22 away from the barrier layer 14 and the perimeter portion of the fastener or adhesive layer 20, thus exposing an outer perimeter of the fastener or adhesive layer 20. The patch 10 is then pressed against the desired substrate, such as a user's skin in a desired location such as those illustrated in FIG. 6. As the patch 10 is pressed against the substrate, the fastener or adhesive layer 20 adhesively attaches the patch 10 to the substrate. If the patch 10 is attached to an immovable substrate such as to an inanimate object, then a defined static area is protected by the vapors which are expressed from the patch 10. If the patch 10 is attached to a movable substrate such as to a person, an animal, or a moving inanimate object, then a movable area in the vicinity of the movable substrate is protected by the insect repellant vapors which emanate from the patch 10.

Referring now to FIGS. 2-4, a structure is shown which is similar to that shown in FIG. 1, except for the addition of a repelling layer 34 located between the pad 12 and the barrier layer 14. In FIG. 2, the lower surface of the pad 12 has a predetermined area and the repelling layer 34 can have approximately the same area. The repelling layer 34 can be adhesively secured to the barrier layer 14. The repelling layer 34 applies a repelling force on either the insect repellant liquid in the pad 12, or on the insect repellant vapors emanating from the pad 12. Where the repelling force affects the liquid, the liquid is urged toward the evaporative interface 36 situated at the top of the pad 12. The evaporative interface 36 is situated between the pad 12 and the carrier layer 16 and represents the location where the liquid is transformed into a vapor upon contact with air. Where the repelling force affects the insect repellant vapors emanating from the pad 12, the rate of removal of vapors from the pad 12 is increased, leaving a lower vapor pressure adjacent the evaporative surface 36, thereby urging an increased rate of evaporation of insect repellant material from the pad 12.

Repelling layer 34 can be fabricated from any material which can impart the desired repelling force. Such force is e.g. an electrostatic force which is imparted to the repelling layer 34 during manufacture of the patch 10 by passing the material of the repelling layer 34 over a discharge device thereby to impart the desired electrical charge.

In some embodiments, the repelling layer 34 is a separate and distinct layer in the patch 10. In other embodiments, the material of the barrier layer 14 is selected such that the properties of the barrier layer 14 perform the repelling function, whereby the repelling function and the barrier function are performed by a single structure. In still other embodiments, the repelling layer 34 is distinct, both in material selection and structure, from the barrier layer 14, but the barrier layer 14 and the repelling layer 34 are fabricated as a single laminate structure whereby the repelling layer 34 and the barrier layer 14 function as a single unitary structure.

In FIG. 2, the general projected size and shape of the repelling layer 34 corresponds to the size and shape of the pad 12, whereby the barrier layer 14 extends outwardly from the outer perimeter of the repelling layer 34. In other embodiments, the projected size and shape of the repelling layer 34 generally corresponds to the projected size and shape of the barrier layer 14. In general, the repelling layer 34 can have any size and shape which is compatible with applying the desired repelling force while staying within the boundaries of the patch 10 consistent with the fastener or adhesive layer 20 being used to attach the patch 10 to a substrate.

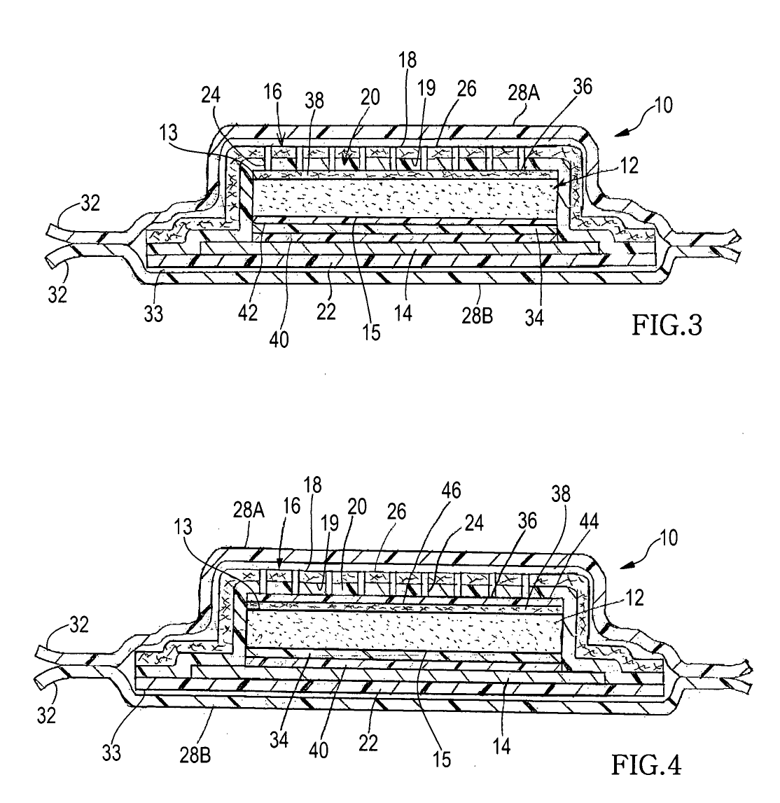

Referring now to FIG. 3, a structure similar to FIG. 1 is shown along with the addition of the repelling layer 34, located between the pad 12 and the barrier layer 14, and the addition of an attractant layer 38. The attractant layer 38 is positioned between the upper surface of the pad 12 and the carrier layer 16 and assists in causing the vapors to exit the patch 10. The attractant layer 38 is directly in contact with the upper surface of the pad 12 and can contain an electrical charge. The electrical charge can produce an electrostatic force which assists the vapors in exiting the patch 10. The attractant layer 38 can be formed with apertures, ports, passageways, interstices, etc. that will allow liquid from the pad 12 to pass completely therethrough. The liquid will be transformed into vapor at the evaporative interface 36 and such vapors can pass out through the vaporization ports 24 formed in the carrier layer 16 and in the fastener or adhesive layer 20. The attractant layer 38 can be formed from a woven or non-woven material that does not require specific apertures, holes or openings to be mechanically formed therein. Alternatively, the attractant layer 38 can be formed with one or more apertures, holes or openings formed therein.

Adhesive layers 40 and 42 can be used to adhesively secure the position of the repelling layer 34 in the patch 10. The adhesive layer 40 attaches the repelling layer 34 to the barrier layer 14. The adhesive layer 42 attaches the repelling layer 34 to the pad 12.

The repelling layer 34 applies a repelling force as in FIG. 2. The attractant layer 38 can apply an attracting electrostatic force to the insect repellant liquid, or can otherwise draw the insect repellant liquid toward the evaporative interface 36 such as by a mechanical wicking action. It should be noted that in this embodiment, the evaporative interface 36 is at the top of the attractant layer 38 rather than at the top of the pad 12, as in the embodiments of FIGS. 1 and 2.

While the structure and composition of the pad 12 is selected to optimize the quantity of liquid held in the pad 12, whereby the pad 12 acts as a liquid reservoir, the structure and composition of the attractant layer 38 is selected and/or designed to draw liquid preferentially from the reservoir/pad toward the top of the pad 12 and into the attractant layer 38. The liquid then moves through the attractant layer 38 to the top of the attractant layer 38 where it leaves the pad/attractant layer combination by evaporating at the evaporative interface 36, and thence moving through the vaporization ports 24 and out of the patch 10. Once out of the patch 10, the insect repellant vapors mingle with the air in the surrounding environment thus to repel insects from that area.

The structure and composition of the attractant layer 38 can be, for example and without limitation, a woven or non-woven layer of natural or synthetic fibers which exerts a mechanical draw on the insect repellant liquid by e.g. capillary action. In some embodiments, the fibers used in the attractant layer 38 have a natural affinity for the liquid of the insect repellant, whereby the combination of the natural affinity of the fibers and the fiber-to-fiber structure of the layer combine to draw the insect repellant liquid toward the evaporative interface 36. The attractant layer 38 can be composed of a meltblown or spunbonded web of polyolefin fibers. The attractant layer 38 can also comprise a carded and/or bonded web composed of natural and/or synthetic fibers.

In a particular embodiment of the present invention, the attractant layer 38 can comprise a spunbonded polypropylene fabric composed of about 2.8-3.2 denier fibers formed into a web having a basis weight of about 22 grams per square meter and a density of about 0.06 grams per cubic centimeter. Such fabric is treated with about 0.3 weight percent of a surfactant.

The attractant layer 38 can be formed from a single layer, or may comprise a multiplicity of components, layers, or partial layers, which correspond to any of the materials disclosed herein for the attractant layer 38, as well as others known in the art.

The attractant layer 38 can be manufactured from a wide selection of web materials, such as porous foams, reticulated foams, apertured plastic films or natural or synthetic fibers. For example, the attractant layer 38 can comprise wood or cotton fibers. Other useful materials are synthetic fibers, such as polyester or polypropylene fibers, or a combination of natural and synthetic fibers.

In some embodiments, the fiber-to-fiber structure encourages the liquid draw but the chemical make-up of the fibers tends to repel the insect repellant liquid. In such embodiments, the fibers are treated with e.g. a surfactant coating which enhances the affinity of the fibers for the insect repellant liquid, thereby to obtain a net draw of insect repellant liquid toward the evaporative interface 36.

Given the combination of the repelling layer 34 and the attractant layer 38 in the embodiment of FIG. 3, the insect repellant liquid experiences both a push force away from the barrier layer 34 and an pull force toward the attractant layer 38, whereby the forces applied by the repelling layer 34 and the attractant layer 38 complement each other in urging movement of the insect repellant liquid toward the evaporative interface 36. As the concentration of insect repellant liquid adjacent the evaporative interface 36 drops in accord with the ongoing evaporation of liquid at the evaporative interface 36, a relatively lower concentration of insect repellant liquid exists adjacent the evaporative interface 36. This concentration gradient acts to urge movement of additional insect repellant liquid toward the evaporative interface 36. In addition, any pushing and pulling forces applied by the repelling layer 34 and the attractant layer 38 enhance the rate of reduction in such concentration gradient, thus urging an incremental increase in concentration of insect repellant liquid at and adjacent the evaporative interface 36.

The attractant layer 38 is shown as a separate layer in the embodiment illustrated in FIG. 3. In other related structures, the attractant layer 38 is physically joined to the pad 12 such as by adhesive attachment, by physically wrapping the attractant layer 38 about the sides of the pad 12, by needling some of the fibers of layer 38 into the pad 12, or the other method of attachment of the attractant layer 38 to the pad 12. In some embodiments, the attractant layer 38 can be mechanically fabricated to the pad 12 such that fibers of the attractant layer 38 extend a substantial distance into the interstices of the pad 12. In such instances, the fibers which so extend into the pad 12 draw liquid from inside the pad 12 whereby the attractant layer 38 draws liquid both from the top surface of the pad 12 and from interior portions of the pad 12 which are spaced from the top surface of the pad 12.

The structure illustrated in FIG. 4 includes a repelling layer 34 which is adhered to the barrier layer 14 by an adhesive layer 40. An attractant layer 38 is located at the top of the pad 12. A control layer 44 is located between the attractant layer 38 and the adhesive layer 20. The control layer 44 is positioned above the attractant layer 38. The control layer 44 can have a predetermined area which is approximately the same as the upper surface of the pad 12. The control layer 44 acts to limit the rate at which vapors of the insect repellant enter the vaporization ports 24, thereby to govern the maximum rate at which vapors leave the patch 10.

The control layer 44 can be formed with apertures, ports, passageways, interstices, etc. that will allow the vapors from the pad 12 to pass completely therethrough and out through the vaporization ports 24 formed in the carrier layer 16 and the fastener or adhesive layer 20. The control layer 44 can be formed from a woven or non-woven material that does not require specific apertures, holes or openings to be mechanically formed therein. Alternatively, the control layer 44 can be formed with one or more apertures, holes or openings formed therein.

When the patch 10 is first opened and the pad 12 has a full charge of insect repellant liquid, in the absence of a control layer 44 with some insect repellant liquid compositions, the full concentration of insect repellant liquid at the evaporative interface 36 can result in an initial surge of evaporation of insect repellant and a corresponding initial surge in vapors which emanate from the patch 10. This initial surge in vapor release is followed by a rapid decay in the rate of release of insect repellant vapors, to a lower relatively steady state rate. In some uses of the patches 10, such a rapid change in rate of release of insect repellant vapors is undesirable.

While choosing to not be bound by theory, applicants contemplate that the initial surge of vaporization causes a substantial reduction in concentration of liquid at the top surface of the attractant layer 38. This in turn, can cause a rapid drop-off in the rate of evaporation over a short period of time. By limiting the maximum rate of evaporation from the evaporation interface 36, embodiments which employ the control layer 44 effectively reduce the amount of vapor released during the initial surge, thereby flattening the rate curve, while not substantially impeding evaporation of the insect repellant liquid during periods of lesser evaporation rates.

The composition of the control layer 44 can be selected from a variety of materials. The structure of the control layer 44 is compatible with the release of vapors, but at a restricted maximum rate. The restriction can be effected by chemical attraction and/or repulsion, or by mechanical limits inherent in the porosity and/or internal mechanical structure of the control layer 44. Thus, there can be recited, as examples, a wide variety of reticulated and/or perforated solid layer structures, as well as woven and/or non-woven structures. Multiple layer structures can be used as desired. The particular structure and composition which are employed in an operable control layer 44 depend on the physical and chemical properties of the insect repellant liquid. When the control layer 44 is formed from a solid film or foil, control of maximum vaporization rate can be established according to the number, size, and distribution of e.g. perforations in the film or foil. The control layer 44 can be formed from a fibrous material which has inherent pores, by virtue of its structure, that can limit or control the passage of vapors therethrough. The structure of the control layer 44 thus controls of maximum vaporization rate. The vaporization rate can be established according to physical parameters, such as and without limitation, the combination of the thickness of the layer, the number of apertures/pores per unit area, the void fraction of the control layer 44, the degree of compaction in the control layer 44, the surface characteristics of the control layer 44, and the like. Typical solid materials are plastic films or metal foils which are perforated to be permissive of passage of the insect repellant vapors at a maximum desired rate. The rate of passage of vapors can be limited by the number of holes, the sizes of the holes, the distribution of the holes, and the locations of the holes in the control layer 44 relative to the locations of the apertures 30 in the carrier layer 16.

Typical materials which are inherently porous are woven and non-woven webs made from natural and/or synthetic fibers, and wherein the web has been consolidated so as to exert effective limits on the rate of passage of vapors. Those skilled in the art will readily select suitable materials from available commercially available webs.

As used herein, “natural fibers” means fibers which occur in nature and which have been extracted from, or released from, naturally-occurring fibrous raw materials, for example vegetative material such as plants and/or trees.

As used herein, “synthetic fibers” means fibers which have been generated from non-fibrous raw materials such as crude oil, natural gas, or oils derived from vegetative material.

In this embodiment, the evaporative interface 36 is at the top of the control layer 44 rather than at the top of the pad 12, as depicted in FIGS. 1 and 2 or at the top of the attractant layer 38 as depicted in FIG. 3.

As in FIGS. 1-3, the structure and composition of the pad 12 is selected to optimize the quantity of liquid held in the pad 12. As in FIGS. 2-3, the structure and composition of the repelling layer 34 is selected and/or designed to exert a pushing force urging the insect repellant liquid toward the top of the pad 12. As in FIG. 3, the structure and composition of the attractant layer 38 is selected and/or designed to draw liquid preferentially from the reservoir/pad toward the top of the pad 12 and into the attractant layer 38. The liquid then moves through the attractant layer 38 to the top of the attractant layer 38 where it passes through the control layer 44 at no more than the maximum rate enabled by control layer 44. The liquid then leaves the control layer 44 by evaporating at the evaporative interface 36 located at the top of the control layer 44. From here, the liquid moves through the vaporization ports 24 and out of the patch 10. As with the previously-discussed embodiments, once the insect repellant vapors exit the vaporization ports 24, the vapors mingle with the air in the surrounding environment thus to repel insects from that area.

Still referring to FIG. 4, because of the combination of the repelling layer 34 and the attractant layer 38, the insect repellant liquid experiences both a push force away from the repelling layer 34 and an pull force toward the attractant layer 38, whereby the forces applied by the repelling layer 34 and the attractant layer 38 complement each other in urging movement of the insect repellant liquid toward the attractant layer 38, and thus toward the evaporative interface 36. This combination push-pull exertion of spaced forces provides a continuous urging of movement of insect repellant liquid toward the attractant layer 38, which provides a desired relatively more constant supply of insect repellant liquid at the interface 46 between the attractant layer 38 and the control layer 44, over the useful life of the patch 10. For a substantial portion of the useful life of the patch 10, the supply of insect repellant liquid at the interface 46 is greater than the supply which can produce the limited rate of vapor release enabled by the control layer 44, whereby the change in rate of release of vapors from the vaporization interface 36 is relatively lower than the rate of change in the absence of use of the control layer 44.

The control layer 44 is shown as a separate layer in the embodiment illustrated in FIG. 4. In other related structures, the control layer 44 is physically joined to the attractant layer 38 such as by an adhesive attachment or the like.

It should be noted that the patch 10 is designed to emits vapor into the atmosphere as liquid in the pad 12 is vaporized by contact with air at the evaporative interface 36. The patch 10 is not designed to adsorb a second liquid while the liquid initially impregnated in the pad 12 is being vaporized. Neither is the patch 10 designed to absorb liquid through the first surface 13 which may be placed against a user's skin. As such, the patch 10 does not function as a wound dressing bandage, wherein body fluid is absorbed into an absorbent pad and any vapors that are present from the absorbed body fluid can emanate out of the bandage. The patch 10 of this invention is designed to allow liquid impregnated in the pad 12 to be transformed into vapor by contact with air and to enable these vapors to emanate from the patch 10.

In general, any material used in the patch 10 must be acceptable for contact with human skin such as by incidental contact. Further, all layers e.g. the carrier layer 16, the fastener or adhesive layer 20, and the release layer 22, which are intended to come into contact with human skin should be benign as to their affects on the human body.

As stated above, the thorough description was explained with a patch 10 designed to contain an insect repellant. However, the patch 10 can contain various liquids which give off vapors that are attractant scents, masking scents or repelling scents. The particular scent can be a perfume, a cologne, a deodorant, an air freshener, for example a pine scent, an outdoor scent, a scent of freshly cut trees, etc, a masking scent for masking almost any imaginable odor, a scent to attract fish or a scent to attract wildlife. A wildlife scent can be a specific vapor for attracting a certain species of animal such as deer, including whitetail deer or mule deer, elk, antelope, bear, turkeys, rabbits, squirrels, etc. A patch 10 containing a scent used to attract a fish or an animal that can be legally caught or hunted can be applied to different objects. For example, a patch 10 containing a fish scent can be attached to a fishing lure or the patch 10 can be incorporated into the lure when the lure is manufactured. The lure as well as the patch 10 can be decorated to look similar to one another. Likewise, a patch 10 containing a hunting scent for attracting a certain species of animal can be mounted or attached to a tree, a stump, a branch, a fence post, a bush, shrubbery etc.

It should also be recognized that a single patch 10 can be individually packaged or multiple patches 10 can be enclosed in a single package. For example, two or more patches 10 can be in a single package. Likewise, multiple patches 10, each designed to give off a different scent, can be sold in a single package. For example, a package of hunting scents may include a number of patches 10 designed to attract deer and a number of patches 10 designed to mask body odor. Each of the patches can be individually wrapped before being enclosed by or inserted into an external package. Another example would be a package of fish scents where one or more of the patches 10 in a single package are designed to attract a certain species of fish. Again, each of the patches 10 can be individually wrapped before being enclosed by or inserted into an external package. A third example is a single package containing patches 10 containing a cologne while other patches 10, within the same package, contain a deodorant. As recited above, each of the patches can be individually wrapped before being enclosed by or inserted into an external package. In the cases where two or more patches 10, one or more of which are designed to emit a different vapor, are present in a single package, the patches 10 can be individually wrapped or sealed before they are inserted into the external package. This will prevent competing odors from intermixing.

### Process

A process for making and packaging the patch 10 will now be described. This process represents only one method of assembling the patch 10 and enclosing it in an external package. Those skilled in the art will recognize that the order of some of the various steps, taught below, can be altered while still arriving with the same end product.

The process includes the steps of placing a liquid impregnated pad 12 onto a barrier layer 14. Alternatively, the pad 12 can be impregnated with a liquid at a later time if so desired. The liquid is capable of vaporizing when contacted by air and the vapors are emitted from the patch 10 as a scent. A fastener 20 is then applied onto or positioned adjacent to a surface of a carrier layer 16 and one or more ports 24, desirably a plurality of ports 24, are formed through both the fastener 20 and the carrier layer 16. The fastener 20 can be an adhesive coating or a layer of adhesive. The carrier layer 16 is then joined to the barrier layer 14 such that the fastener 20 faces the pad 12 and at least a portion of the barrier layer 14. A release layer 22 is then applied below the barrier layer 22. The size of the release layer 22 can be larger than the barrier layer 14 such that a portion of the release layer 22 is removably secured to the fastener 20. For example, the fastener and the release layer 22 can encircle the periphery of the barrier layer 14. The patch 10 is then enclosed and/or sealed between two over wrap layers 28A and 28B and/or the patch 10 can be enclosed in a conventional external package. It should be noted that the two over wrap layers 28A and 28B can be replaced by another structure that will serve the same function. That function is to enclose and retain the patch 10 in a liquid impermeable and vapor impermeable setting.

Optionally, a repelling layer 34 can be positioned above the barrier layer 14 to assist in emitting vapors from the pad 12. Another alternative is to position an attractant layer 38 above the pad 12 to assist in emitting vapors from the pad 12.

Those skilled in the art will see that certain modifications can be made to the apparatus and methods herein disclosed with respect to the illustrated embodiments, without departing from the spirit of the instant invention. While the invention has been described above with respect to the illustrated embodiments, it will be understood that the invention is adapted to numerous rearrangements, modifications, and alterations, and all such arrangements, modifications, and alterations are intended to be within the scope of the appended claims.

To the extent the following claims use means plus function language, it is not meant to include there, or in the instant specification, anything not structurally equivalent to what is shown in the embodiments disclosed in the specification.

I claim:

1. A patch capable of dispensing a vapor, comprising:
a) a pad impregnated with a liquid which is capable of being vaporized, said pad having a first surface and a second surface;
b) a carrier layer positioned above said first surface of said pad, said carrier layer having an upper surface and a lower surface with said upper surface forming a top outside surface of said patch, and said carrier layer having a periphery;
c) a barrier layer positioned below said second surface of said pad, and said barrier layer having a periphery smaller than the periphery of said carrier layer;
d) a fastener secured to at least a portion of said lower surface of said carrier layer, said fastener cooperating with said barrier layer to form a lower surface of said patch, and said fastener being capable of releasably attaching said patch to another object;
e) a release layer positioned below said barrier layer and being removably secured to said fastener, said release layer being removed before said patch is used;
f) an evaporative interface situated between said pad and said carrier layer where said liquid is transformed into a vapor upon contact with air; and
g) a plurality of vaporization ports extending from said evaporative interface to said upper surface of said carrier layer through which such vapors can pass.

2. The patch of claim 1 wherein said evaporative interface is a demarcation below which liquid exists in said pad and above which vapor exists in said carrier layer, and wherein said pad is formed from at least one layer which contains hydrophilic fibers.

3. The patch of claim 1 wherein said fastener is an adhesive.

4. The patch of claim 3 wherein each of said plurality of vaporization ports forms a continuous passageway through both said adhesive and said carrier layer, and each of said plurality of vaporization ports has a restrictive opening through which only vapors can pass.

5. The patch of claim 4 wherein said pad contains areas of higher liquid holding capacity and areas of lower liquid holding capacity, and said liquid is unable to pass through said plurality of vaporization ports.

6. The patch of claim 1 wherein said carrier layer and said barrier layer are both formed from a liquid impermeable and vapor impermeable material, and said plurality of vaporization ports transforms said carrier layer into a vapor permeable material once said plurality of vaporization ports are formed therein.

7. The patch of claim 6 wherein said carrier layer is a metal foil and said barrier layer is polyethylene.

8. The patch of claim 1 wherein said release layer has a periphery essentially coextensive with the periphery of said carrier layer and said release layer is designed to be removed prior to attaching said patch to an object.

9. The patch of claim 1 wherein said liquid is a masking scent such as a perfume, a cologne, a deodorant; a repellant scent such as an insect repellant; or an attractant scent such as a fish scent or a wild game scent.

10. A patch capable of dispensing a vapor containing a scent, comprising:
a) a pad impregnated with a liquid which is capable of being vaporized, said pad having a first surface and a second surface;
b) a carrier layer positioned above said first surface of said pad, said carrier layer having an upper surface and a lower surface with said upper surface forming a top outside surface of said patch, and said carrier layer having a periphery;
c) a barrier layer positioned below said second surface of said pad, and said barrier layer having a periphery smaller than the periphery of said carrier layer;
d) an adhesive secured to said lower surface of said carrier layer, said adhesive cooperating with said barrier layer to form a lower surface of said patch, and said adhesive being capable of releasably attaching said patch to another object;
e) a repelling layer positioned between said lower surface of said pad and said barrier layer, said repelling layer assisting in causing such vapors to exit said patch;
f) a release layer positioned below said barrier layer and being removably secured to at least a portion of said adhesive, said release layer preventing said adhesive from becoming contaminated prior to attachment of said patch to another object;
g) an evaporative interface situated between said pad and said carrier layer; and
h) a plurality of vaporization ports extending from said evaporative interface to said upper surface of said carrier layer, said plurality of vaporization ports enabling such vapors to exit said patch.

11. The patch of claim 10 wherein said lower surface of said pad has a predetermined area and said repelling layer has approximately the same area.

12. The patch of claim 11 wherein said repellant layer is directly in contact with said lower surface of said pad and said repelling layer contains an electrical charge, which in turn produces an electrostatic force which assist said vapors in exiting said patch.

13. The patch of claim 12 wherein said repelling layer is adhesively secured to barrier layer.

14. The patch of claim 10 further comprising an attractant layer positioned between said upper surface of said pad and said carrier layer, said attractant layer assisting in causing such vapors to exit said patch.

15. The patch of claim 14 wherein said attractant layer is directly in contact with said upper surface of said pad and said attractant layer contains an electrical charge, said electrical charge capable of producing an electrostatic force which assists such vapors in exiting said patch.

16. A patch capable of dispensing a vapor containing a scent, comprising:
a) a pad impregnated with a liquid which is capable of being vaporized, said pad having a first surface and a second surface;
b) a carrier layer positioned above said first surface of said pad, said carrier layer having an upper surface and a lower surface with said upper surface forming a top outside surface of said patch, and said carrier layer having a periphery;
c) a barrier layer positioned below said second surface of said pad, and said barrier layer having a periphery smaller than the periphery of said carrier layer;
d) an adhesive secured to said lower surface of said carrier layer, said adhesive cooperating with said barrier layer to form a lower surface of said patch, and said adhesive being capable of releasably attaching said patch to another object;
e) a repelling layer positioned between said lower surface of said pad and said barrier layer, said repelling layer assisting in causing such vapors to exit said patch;
f) an attractant layer positioned between said upper surface of said pad and said carrier layer, said attractant layer assisting in causing such vapors to exit said patch;
g) a release layer positioned below said barrier layer and being removably secured to at least a portion of said adhesive, said release layer preventing said adhesive from becoming contaminated prior to attachment of said patch to another object;
h) an evaporative interface situated between said pad and said carrier layer; and
i) a plurality of vaporization ports extending from said evaporative interface to said upper surface of said carrier layer, said plurality of vaporization ports enabling said vapors to exit said patch.

17. The patch of claim 16 further comprising a control layer positioned between said upper surface of said pad and said carrier layer, said control layer capable of regulating the amount of such vapors that can initially exit said patch.

18. The patch of claim 16 wherein said control layer is positioned above said attractant layer.

19. The patch of claim 16 wherein said upper surface of said pad has a predetermined area and said control layer has approximately the same area.

20. The patch of claim 16 wherein said patch is housed in a liquid impermeable and vapor impermeable package.

21. A patch having a bottom outside mounting surface and an opposing top outside surface, said patch comprising:
a) a carrier layer generally defining said top outside surface;
b) a barrier layer disposed toward said bottom outside surface;
c) a pad having a liquid therein, said pad being positioned between said barrier layer and said carrier layer;
d) an evaporative interface located between said pad and said carrier layer;
e) a plurality of vaporization ports which extend as continuous passages between said evaporative interface and said top outside surface; and
f) at least one of a mounting adhesive and a mounting structure being disposed at said bottom outside surface for attaching said patch to another object.

22. A flexible insect repellant dispenser having a bottom outside mounting surface and an opposing top outside surface, said flexible insect repellant dispenser comprising:
a) a carrier layer generally defining said top outside surface;
b) a barrier layer disposed toward said bottom outside surface;
c) a pad having a liquid therein, said pad being positioned between said barrier layer and said carrier layer;
d) an evaporative interface located between said pad and said carrier layer;
e) a plurality of vaporization ports which extend as continuous passages between said evaporative interface and said top outside surface; and
f) at least one of a mounting adhesive and a mounting structure being disposed at said bottom outside surface for attaching said insect repellant dispenser to another object.

23. The patch of claim 21 wherein in use, said patch is adapted to be attached to an object such that said barrier layer lies adjacent to said object and said carrier layer is facing away from said object.

24. The patch of claim 21 wherein in use, said patch emits vapor into the atmosphere as liquid in said pad is vaporized by contact with air and said pad is not able to absorb a second liquid through said carrier layer while said liquid initially impregnated in said pad is being vaporized.

25. A package containing at least two patches as described in claim 21.

26. The package of claim 25 wherein each patch is individually wrapped within said package.

27. The package of claim 25 wherein one patch contains an attractant scent and another patch contains a masking scent.

28. The package of claim 27 wherein said attractant scent is cologne and said masking scent is a deodorant.

29. The package of claim 25 where one patch contains a scent for attracting a first species of fish and another patch contains a scent for attracting a second species of fish.

30. A package containing multiple patches, at least one of said patches containing a scent for attracting a first species of animal and a second patch containing a scent for attracting a second species of animal.

31. A package containing multiple patches, at least one of said patches containing a scent for attracting a first species of animal, a second patch containing a scent for attracting a second species of animal, and a third patch containing a scent for attracting a third species of animal.

32. A process for making a patch comprising the steps of:
a) placing a liquid impregnated pad onto a barrier layer, said liquid being capable of emitting vapors when contacted by air, and such vapors emitting a scent;
b) applying a fastener onto a surface of a carrier layer and forming a plurality of ports through both said fastener and said carrier layer;
c) joining said carrier layer to said barrier layer such that said fastener faces at least a portion of said barrier layer and said pad is located between said carrier layer and said barrier layer; and
d) applying a release layer below said barrier layer such that said barrier layer is between said pad and said release layer, and wherein a portion of said release layer is removably secured to said fastener.

33. The process of claim 32 further comprising positioning a repelling layer above said barrier layer to assist in emitting vapors from said pad, said repelling layer being located between said pad and said barrier layer.

34. The process of claim 32 further comprising positioning an attractant layer between said pad and said carrier layer to assist in emitting vapors from said pad.

35. The process of claim 34 further comprising forming openings in said attractant layer to enable such vapors to pass completely therethrough.

36. The process of claim 34 further comprising positioning a control layer outwardly of said attractant layer such that said attractant layer is located between said control layer and said pad, said control layer being capable of regulating the passage of vapors from said patch.

37. The process of claim 36 further comprising forming openings in said control layer to enable vapors to pass completely therethrough.

38. The process of claim 32 wherein said patch is enclosed in a wrapper.

39. The process of claim 38 further comprising constructing said wrapper from at least one liquid impermeable material.

40. The process of claim 38 further comprising constructing said wrapper from at least one vapor impermeable material.

41. The process of claim 38 further comprising constructing said wrapper from at least one liquid impermeable and vapor impermeable material.
